(19) United States
(12) Patent Application Publication
(10) Pub. No.: US 2003/0108964 A1
(43) Pub. Date: Jun. 12, 2003

(54) Caspase-inhibitory-factor(cif)and uses thereof

(76) Inventor: Andrea Leblanc, Quebec (CA)

Correspondence Address: WOOD, PHILLIPS, KATZ, CLARK & MORTIMER, 500 W. MADISON STREET, SUITE 3800, CHICAGO, IL, 60661, US

(21) Appl. No.: 10/220,515

(22) PCT Filed: Feb. 21, 2001
(86) PCT No.: PCT/CA01/00210

(51) Int. Cl. G01N 33/574; C12N 9/99

(52) U.S. Cl. 435/7.23; 435/184

(57) ABSTRACT

The present invention relates to the identification of a caspase inhibitory factor (CIF) and to the establishment of a screening procedure to find caspase inhibitors in human neurons. More particularly, the invention relates to a 17-β-estradiol inducible caspase-6 inhibitory factor.

### BACKGROUND OF THE INVENTION

(a) Field of the Invention

The invention relates to the identification of a caspase inhibitory factor (CIF) and to the establishment of a screening procedure to find caspase inhibitors in human neurons. More particularly, the invention relates to a 17-β-estradiol inducible caspase-6 inhibitory factor.

(b) Description of Prior Art

Caspases are a group of cysteinyl proteases with substrate specificities for aspartic acid. There are 14 mammalian caspases ranging in size from 32-55 kDa (Nicholson D (1999) Cell Death and Differentiation 6:1028-1042). Caspases are activated by proteolytic processing of a pro-arm N-terminal fragment and by endoproteolytic processing to create two fragments of approximately 10 (p10) and 20 (p20 kDa). Two molecules of each assemble to form the active tetrameric enzyme. Each caspase has substrate preferences for four amino acid sequence substrates although high levels of caspase activity may result in promiscuity amongst caspase substrates.

There are two types of caspase inhibitors, natural and synthetic. Natural inhibitors of caspase-6 are unknown at this time. The activity of other caspases are inhibited by six different groups of natural inhibitors; viral inhibitors, inhibitor of apoptosis proteins (IAPs), caspase-specific decoy molecules, nitric oxide, Bcl-2 proteins and phosphorylation (Ekert P et al. (1999) Cell Death and Differentiation 6:1081-1086). Viral inhibitor, Cowpox virus product cytokine response modifier A (Crm A), prevent caspase activity by direct interaction with the pro-enzyme thus preventing its proteolytic activation. Baculoviral protein p35 is cleaved by caspases and the cleaved subunits of p35 form an inhibitory complex with caspases. Crm A inhibits caspase-1 and -8 but not -3, -6, or -7 while p35 can inhibit caspase-1, -3, -6, -7, -8, and -10. Members of the IAP family, X-IAP, c-IAP-1, c-IAP-2, and N-AIP, inhibit caspases-3, -7, and -9 by direct interaction with the caspases but none can inhibit caspase-6. Decoy or mimic protein inhibitors such as FLICE and ARC and maybe truncated Csp-9 prevent activation of the pro-. Similar to truncated caspase-9, Mch2β, may act as a competitive inhibitor of caspase-6 activation. Nitric oxide nitrosylation of the cysteine residues of caspase-3 inhibits activity and inhibition is reversible by 20 mM DTT. In addition, anti- and pro-apoptotic members of the Bcl-2 family of proteins interact with caspase-9 co-activator, Apaf-1, and modulate caspase-9 activity. Phosphorylation of pro-caspase-9 or the large subunit of caspase-9 by serine/threonine kinase, Akt, inhibits caspase-9 activity. In summary, Bcl-2, phosphorylation and mimic or decoy molecules inhibit the activation of pro-caspases while CrmA, p35, IAPs, phosphorylation and nitrosylation inhibit the active form of caspases. CIF activity cannot be due to p35 since this is a viral gene, and IAPs do not inhibit caspase-6. In preliminary data, we provide evidence against nitrosylation. Akt phosphorylation is unlikely since caspase-6 lacks consensus Akt phosphorylation motifs. Therefore, we believe that CIF is a novel 17-β-estradiol regulated inhibitor that acts directly on the active form of caspase-6.

Synthetic peptide inhibitors are made based on the specificity of caspases for four amino acid substrates with an obligatory aspartic acid at P1. Classification of caspases have been established based on substrate preference (Thornberry N A (1999) Cell Death and Differentiation 6:1023-1027). While synthetic caspase inhibitors hold great promise for many disease, there is a concern that they may not target specific cell types and in the case of the brain could predispose to tumorigenicity rather than simply prevent neuronal cell death by caspase inactivation. Therefore, it is essential to understand the natural mechanism of caspase inactivation in neurons in order to establish therapies that target a cell type specific mechanism rather than a general one.

Caspases are involved in physiological and non-physiological neuronal apoptosis. Non-physiological cell death occurs in many neurodegenerative diseases such as Alzheimer's disease, Parkinson's disease, amyotropic lateral sclerosis (ALS), cerebellar degeneration, ischemia (stroke), traumatic injuries, prion diseases, Huntington disease. Apoptosis in other tissues also leads to human diseases. These include osteoporosis, myocardial infarction or other cardiovascular diseases and chronic inflammation such as rheumatoid arthritis and acute inflammation. Furthermore, induction of cancer is associated with a dysregulation of normal cell death. Estrogen is known to protect against Alzheimer's disease and osteoporosis and induce breast and uterine cancer. Therefore, inhibitors of caspases can be applied to protect against apoptotic diseases and down regulation of inhibitors of caspases can be used to prevent or diminish tumor formation.

Epidemiological studies have shown that decreasing levels of estrogen is a risk factor for Alzheimer's disease and hormone replacement therapy with estrogen (HRT) offers some protection against Alzheimer's disease (Paganini-Hill A (1996) Br. J. Obs. Gyn. 103:80-86) Further evaluation of the potential role of estrogen on neurons (Woolley C S (1999) Curr. Opin. Neurobiol. 9:349-354) identified that estrogen enhances neuritic outgrowth and survival, upregulates brain derived neurotrophic factor, nerve growth factor and epidermal growth factor, and reverses the behavioral and biochemical changes in ovariectomized rats. In addition, estrogen decreases the amount of amyloid β peptide produced in neurons and can protect against amyloid β peptide mediated neurotoxicity. Estrogen modulates p53 activity and cell fate, and the expression of Bcl-2 proteins. Others propose that estrogen acts as an anti-oxidant although it is unlikely that physiological levels of estrogen will have antioxidant activity. None of these studies have found inhibition of caspase-mediated cell death by estrogens.

Estrogen has a wide variety of effects on different cellular mechanisms. In this section, I focused only on those mechanisms that are potentially involved in neuronal survival or cell death. There are two estrogen receptors, ER-α and ER-β. Both are expressed in brain in neurons and in astrocytes. Estrogens modulates cellular activities through receptor-mediated nuclear gene transcriptional activation or through non-genomic mechanism via signal transduction pathways. Binding of estrogen to its receptor initiates transcriptional gene expression in estrogen responsive element and estrogen-responsive AP1 enhancer containing genes (reviewed by Woolley C S (1999) Curr. Opin. Neurobiol. 9:349-354). While both ER-α and ER-β act on ERE-responsive genes, ER-β modulates the activity of estrogen responsive AP1 elements. Survival genes containing EREs include Bcl-2 or Bcl-xL and BDNF. These effects could explain part of the role of estrogen in neuroprotection. Bcl-2 proteins can inhibit pro-caspase activation but not active enzyme. Therefore, CIF which acts on the active caspase-6 cannot be a Bcl-2 protein. We have also eliminated the possibility that CIF is actively translated in 17-β-estradiol treated neurons since the presence of cycloheximide does not inhibit CIF activity. In addition, CIF activity occurs as early as 10 minutes after 17-β-estradiol treatment indicating that a non-genomic signal transduction mechanism is responsible for CIF activity.

Caspase-6 (Mch2α) is a member of the group of cysteine-dependent aspartate specific proteases that are critically involved in apoptotic cell death (reviewed by Nicholson D (1999) Cell Death and Differentiation 6:1028-1042). Caspase-6 is an effector short-arm pro-enzyme that is proteolytically activated by caspase-1, 3, -7, -8 and -11. Once activated, caspase-6 cleaves endogenous substrate proteins such as lamin A and amyloid precursor protein (LeBlanc A C et al. (1999) J. Biol. Chem. 274:23426-23436). We have shown that serum deprivation mediated neuronal cell death activates caspase-6 (LeBlanc A C et al. (1999) J. Biol. Chem. 274:23426-23436). In addition, caspase-6 alters amyloid precursor protein metabolism and increases production of amyloid β peptide. Furthermore, caspase-6 but not caspase-3, 7, and 8 induce a protracted course of selective neuronal apoptosis in human neurons (Zhang Y et al. (2000) J. Neurosci. 20:8384-8389). Caspase-6 p10 fragments generated through activation of caspase-6 are increased in Alzheimer's disease brains and suggest that caspase-6 may play an important role in the pathogenesis of Alzheimer's disease (LeBlanc A C et al. (1999) J. Biol. Chem. 274:23426-23436). Therefore, it is of interest to determine if natural inhibitors of caspase-6 exist in these human neurons.

Neuronal inhibitors of active caspase-6 are unknown at this time. The activity of other caspases are inhibited by six different groups of inhibitors; viral inhibitors, inhibitor of apoptosis proteins (IAPs), caspase-specific decoy molecules, oxidative agents, Bcl-2 proteins and phosphorylation (reviewed by Ekert P et al. (1999) Cell Death and Differentiation 6:1081-1086). Bcl-2, decoy or mimic protein inhibitors such as FLICE and ARC, truncated caspase-9 (Csp-9), Mch-2 beta, IAPs and phosphorylation of caspase-9 can prevent activation of the pro-enzyme form of caspases. Cowpox virus product cytokine response modifier A (Crm A), baculoviral protein p35, IAPs, nitric oxide nitrosylation and selenium oxidation inhibit the active caspases. p35 can inhibit caspase-6 but none of the other inhibitors including IAPs were shown to inhibit caspase-6 activity.

It would be highly desirable to be provided with the identification of a caspase inhibitory factor (CIF) and to the establishment of a screening procedure to find caspase inhibitors in human neurons.

### SUMMARY OF THE INVENTION

One aim of the present invention is to provide a 17-β-estradiol induced caspase inhibitory factor (CIF) with activity against caspase-6 mediated neuronal cell death.

Another aim of the present invention is to provide a 17-β-estradiol induced caspase inhibitory factor in neurons with activity against endogenous and recombinant caspase-6.

Another aim of the present invention is to provide a 17-β-estradiol induced caspase inhibitory factor that does not require de novo protein synthesis.

Another aim of the present invention is to provide 17-β-estradiol induced caspase inhibitory factor that is not nitric oxide.

Another aim of the present invention is to provide a 17-β-estradiol induced caspase inhibitory factor in human breast cancer cell line, MCF7.

Another aim of the present invention is to provide a screening method for screening a variety of drugs capable of inducing or inhibiting CIF in human neurons or other estrogen-responsive tissues.

In accordance with the present invention there is provided a caspase inhibitory factor (CIF) which comprises a factor endogenous to a human primary culture of neurons and endogenous to a human breast cancer cell line, MCF-7, and wherein said CIF being inducible by 17-β-estradiol and being capable of preventing apoptosis and/or synaptic degeneration.

The prevention of apoptosis and/or of synaptic degeneration may be effected by inhibiting at least one caspase, such as for example caspase-3, -6, -7, and -8.

In accordance with the present invention there is provided a drug screening assay for potential neuronal inhibitors of caspases, which comprises using a caspase-inhibitory-factor (CIF) of the present invention to test for compounds capable of activating CIF

In accordance with the present invention there is provided a drug screening assay for potential compounds for the treatment of neurodegenerative diseases and metabolic bone diseases, which comprises using caspase-inhibitory-factor (CIF) of the present invention to test for compounds capable of activating CIF.

The metabolic bone diseases are selected from the group consisting of osteomalacia, osteoporosis, osteopetrosis and Paget's disease.

The neurodegenerative diseases are selected from the group consisting of Parkinson's, Alzheimer's disease, neuronal loss associated with dementia, amyotropic lateral sclerosis (ALS), cerebellar degeneration, ischemia (stroke), traumatic injuries, prion diseases and Huntington disease.

In accordance with the present invention there is provided a drug screening assay for potential compounds for the treatment of estrogen responsive cancers, which comprises using caspase-inhibitory-factor (CIF) of the present invention to test for compounds capable of inhibiting CIF.

The estrogen responsive cancers are breast and uterine cancer.

In accordance with the present invention there is provided a method for the treatment of neurodegenerative diseases and metabolic bone diseases, which comprises administering an effective amount of a compound capable of activating CIF.

In accordance with the present invention there is provided a method for the treatment of estrogen responsive cancers which comprises administering an effective amount of a compound capable of inhibiting CIF.

In accordance with the present invention there is provided a method for the protection against apoptosis in estrogen responsive tissues in a patient, which comprises administering an effective amount of an estrogen compound capable of activating CIF.

The estrogen responsive tissues comprises neurons and bone.

In accordance with the present invention there is provided a method of determining the ability of cells to become malignant, which comprises determining whether the presence of estrogen in said cells increases the activity CIF.

In accordance with the present invention there is provided a method of diagnosis of a disease associated with apoptosis, which comprises detecting and/or quantitating CIF activity.

### BRIEF DESCRIPTION OF THE DRAWINGS

FIG. 1 illustrates that 17-β-estradiol inhibits R-Csp-6-mediated apoptosis. A. Neurons were injected with 5 pg/cell of R-Csp-6 and 0.1 ng dextran Texas red and treated in absence (positive control) or presence of 10 nM of each of the indicated hormones or the equivalent amount of ethanol used to dissolve the hormones (ethanol control). The ethanol and negative controls were microinjected with caspase-6 active buffer and dextran Texas Red. TUNEL was performed after 48 hours of incubation. Data represents the mean and SD of three independent experiments. p<0.0001 for 17-β-estradiol and >0.1 for other hormones. B. Neurons were microinjected with 5 pg/cell of R-Csp-6 and incubated in the absence (control) or presence of 10 nM 17-β-estradiol. At the indicated time point, cells were fixed and processed for TUNEL. Data represents the mean and SEM of 2-3 assays for each of three independent neuron preparations. p<0.06 at 1 day and p<0.0001 from 2-16 days. C. Neurons were microinjected with 5 pg/cell of R-Csp-6 and treated with varying concentrations of 17-β- and 17-α-estradiol. Data represents the mean and SEM of 4 experiments. *p<0.05 for 1-100 nM of 17-β-estradiol.

FIG. 2 illustrates that the recombinant caspase-6 activity is inhibited with the addition of neuronal extracts from 17-β-estradiol-treated neurons. A. In vitro caspase-6 activity in the presence of 10 μg protein from untreated (control), 17-β-estradiol or 17-β-estradiol-treated neuronal extracts at the indicated concentration. Neurons were treated for 6 hours. The control represents untreated neuronal extract and was arbitrarily placed at 100%. *p<0.02. B. Dose-dependent inhibition of recombinant active caspase-6 in vitro with the addition of indicated amounts of 17-β-estradiol-treated neuronal proteins. p<0.008 from 0.5 to 10 μg. C. Inhibition of endogenous neuronal caspase-6 in 17-β-estradiol-treated neurons for 48 hours. Data represents the mean and SEM from three independent neuron preparations. p<0.01.

FIG. 3 illustrates the rapid induction of CIF by 17-β-estradiol. A. Time course of CIF induction at 10 minutes and 1, 6, 12, 24 and 48 hours. p<0.03 from 10 minutes to 24 hours. Data represent the mean and SD of four independent experiments. B. Neurons were treated with 17-β-estradiol treatment for indicated time, the hormone washed away and cell further incubated until 48 hours. TUNEL was used to measure neuronal cell death. Data represent the mean and SEM of three independent experiments. p<0.001 from 10 minutes to 48 hrs.

FIG. 4 illustrates that induction of CIF by 17-β-estradiol does not require de novo protein synthesis. A. Autoradiogram of total cellular and immunoprecipitated secreted amyloid precursor protein from neurons radiolabeled in the presence or absence of cycloheximide at 5 or 20 μg/ml. B. Neuroprotective effect of 17-β-estradiol in the absence or presence of cycloheximide. No significant difference was obtained (p>0.1). C. CIF activity in 17-β-estradiol-treated neurons in the absence or presence of cycloheximide. p<0.05 between untreated and 17-β-estradiol but no significant difference is obtained with cycloheximide (p>0.9). Data for B & C represent the mean and SD of four independent experiments.

FIG. 5 illustrates the mechanisms of CIF activation. A. CIF is not inhibited by DTT. CIF containing neuronal extracts were assayed in the presence of 10 or 20 mM DTT. Data represents the mean and SEM of three independent experiments. p>0.83 between 10 and 20 mM DTT in 17-β-estradiol treated extracts and p<0.008 between untreated and 17-β-estradiol treated protein extracts. B. Tamoxifen antagonizes 17-β-estradiol mediated neuroprotection. Neurons were microinjected with 5 pg/cell of R-Csp-6 and incubated in 10 nM 17-β-estradiol in the absence or presence of 10 μM tamoxifen. Data represents the mean and SEM of three independent experiments. C. Tamoxifen antagonizes 17-β-estradiol induction of CIF. Neurons were treated with 10 nM 17-β-estradiol in the absence or presence of 10 μM tamoxifen. Neuronal extracts were assayed for CIF activity. Data represents the mean and SEM of four independent experiments.

FIG. 6 illustrates the CIF activity on caspase-3, 7, and 8. Neuronal extracts containing CIF activity against R-Csp-6 were tested for inhibitory activity of caspase-3, 7, and 8. Results show the mean and SEM of three independent experiments. The control represents neuronal extracts from untreated neurons. The third column represents the activity of the recombinant caspase in absence of neuronal protein extract. p<0.02 for caspase-6 and p<0.00007 for caspase-3, -7, and -8.

FIG. 7 illustrates the CIF activity in astrocytes. A. Human astrocytes extracts were tested for CIF activity after a 6 hour treatment with 17-β-estradiol. B. Human astrocytes were microinjected with caspase-3 and incubated in the presence or absence of 10 nM 17-β-estradiol. Cell death was measured by TUNEL. No significant difference is observed between untreated and 17-β-estradiol treated astrocytes.

FIG. 8 illustrates that diethylstilbesterol and ethinyl estradiol act as antagonists of estrogen receptor-mediated induction of CIF. A. Neurons were treated with 10 nM DES, EE, or tamoxifen in the presence or absence of 17-β-estradiol. Neuronal extracts were tested for CIF activity. B. Neurons were microinjected with DTR and 5 pg/cell of R-Csp-6 and incubated with 10 nM DES, EE, or tamoxifen in the absence or presence of 17-β-estradiol for 48 hours. Cell death was determined by TUNEL. These data represent the mean and SEM of three independent experiments.

TABLE I
CIF is induced by 17-β-estradiol in human breast cancer
cell line, MCF7

| | | | R- Csp-6 | |
|---|---|---|---|---|
| | | | Specific | % |
| MCF7 extract | Endogenous Csp-6 | % | Activity | Inhibition |
| treated 48 hours with | Specific Activity | Inhibition | nmoles/μg | of R- |
| 17- -estradiol. | pmoles/μg protein/min. | of Csp-6 | protein/min. | Csp-6 |
| Control | 6.34 (0.02) | 100% | 6.15 (0.7) | 100% |
| 1 nM 17-β-estradiol | 4.02 (0.02) | 63% | 3.82 (0.04) | 62% |
| 10 nM 17-β-estradiol | 4.84 (0.03) | 76% | 3.25 (0.03) | 53% |
| 100 nM 17-β-estradiol | 5.06 (0.01) | 80% | 4.30 (0.1) | 70% |
| 10 nM 17-α-estradiol | 7.13 (0.06) | 112% | 7.19 (0.01) | 117% |

To determine if estrogen-mediated CIF activity could be responsible for estrogen-responsive cancer cell lines and also to find a cell line for the purification and identification of CIF activity, we tested the effect of 17-β-estradiol on the human estrogen-responsive breast cancer cell line, MCF7. The results show that of 17-β-estradiol but not of 17-α-estradiol induce CIF activity against endogenous or exogenous caspase-6 (Table I).

### DETAILED DESCRIPTION OF THE INVENTION

Surprisingly and in accordance with the present invention, it is demonstrated that 17-β-estradiol but not 17-α-estradiol, testosterone, or epitestosterone delay caspase-6 mediated neuronal cell death. 17-β-estradiol-treated neuronal extracts directly inhibit recombinant active caspase-6 in an in vitro assay. We conclude that 17-β-estradiol induces a caspase inhibitory factor (CIF) that is preventing neuronal apoptosis. The effect is antagonized by estrogen receptor antagonist, tamoxifen. In contrast, 17-β-estradiol does not induce CIF nor prevent caspase-mediated cell death in astrocytes. The induction of CIF occurs within 10 minutes of neuronal exposure to 17-β-estradiol and does not require de novo protein synthesis. CIF is a broad spectrum caspase inhibitor. CIF is not acting through oxidation of the caspase active site. Furthermore, diethylstilbesterol and ethinyl estradiol cannot induce CIF in neurons but antagonize 17-β-estradiol induction of CIF. The present results indicate that 17-β-estradiol induces a novel inhibitor of active caspases through estrogen receptors and provide an additional mechanism for the neuroprotective action of 17-β-estradiol. This mechanism is likely highly relevant to the understanding of the role of estrogen against Alzheimer's disease.

To determine if active caspase-6 leads to an obligatory neuronal cell death or can be inhibited, we assessed various known neuroprotective agents against caspase-6-mediated cell death. In accordance with the present invention, we describe a role for 17-β-estradiol against caspase-6-mediated apoptosis. It is well established that women on hormone replacement therapy are at a lower risk for Alzheimer's disease (Paganini-Hill A (1996) Br. J. Obs. Gyn. 103:80-86). The neuroprotective role of estrogen has been attributed to a genomic dependent mechanism possibly through the expression of Bcl-2 proteins. We show that co-treatment of caspase-6 microinjected neurons with physiological amounts of 17-β-estradiol protects the neurons against apoptosis. Neuronal extracts from 17-β-estradiol treated neurons inhibit recombinant caspase-6 activity in vitro. Our results indicate that 17-β-estradiol induces a caspase inhibitory factor (CIF) through a non-genomic pathway. Furthermore, these results introduce a novel regulatory mechanism of caspases that have important implications for the modulation of human neuronal cell death by 17-β-estradiol.

Cell Cultures: Primary Cultures of Neurons and Astrocytes

Primary cultures of neurons were established from 12-14 week old foetal brains, according to ethical regulations of the Medical Research Council of Canada and approved by McGill University. Institutional Review Board. Briefly, cortical and subcortical brain tissue is minced, dissociated in 0.25% trypsin for 15 minutes at 37° C. Trypsin is inactivated with 10% serum and 0.1 mg/ml deoxyribonuclease I added before triturating to completely dissociate the cells. The mixture is successively passed through 130 μm and 70 μm filters, and cells plated at 3×10⁶/ml on poly-L-lysine coated tissue culture dishes or ACLAR™ (33C; 5 mm; Allied Chemical Corp.) coverslips in phenol-free minimal essential media in Earle's balanced salt solution containing 0.225% sodium bicarbonate, 1 mM sodium pyruvate, 2 mM L-glutamine, 0.1% dextrose, 1×antibiotic Pen-Strep (all products from Gibco-BRL) and 5% decomplemented fetal bovine serum (Hyclone). In serum, testosterone is present at a final concentration of 9 pM and estrogen is 18 pM.

The cells attach rapidly and establish intricate neuritic networks within 3 days. Fluorodeoxyuridine (FdU) is added at 1 mM to prevent proliferation of dividing cells. Typically, the culture is composed of 90-95% neurons and 5-10% astrocytes that survive in culture for 4-6 weeks. Experiments on neurons and astrocytes were conducted at 10 days of culture.

Microinjection of Recombinant Caspase-6 in Neurons or Caspase-3 in Astrocytes

Glass micropipettes of 1.0 mm OD and 0.5 mm ID thin-walled glass capillaries with microfilaments (Borosilicate with filament MTW100F-4, World Precise Instrument Co.) were pulled with a Flaming/Brown micropipette puller (P-87) with a tip diameter of −0.51 μm. Recombinant active caspase-6 or caspase-3 (R-Csp-6 and R-Csp-3 from Pharmingen) were prepared in caspase active buffer containing 20 mM piperazine-N,N′-βis-(2-ethanesulfonic acid) (PIPES), 100 mM NaCl, 10 mM dithiothreitol (DTT), 1 mM EDTA, 0.1% 3-[(3-cholamidopropyl)-dimethylammonio]-2-hydroxy-1-propanesulfonic acid (CHAPS), 10% sucrose, pH 7.2. R-Csp-6 was co-injected with Dextran Texas Red (DTR; at 100 μg/ml) (Cedarlane Laboratories Ltd.) as a fluorescent marker to recognize injected neurons. Control injections contain DTR and caspase-6 active buffer. Microinjections were done with the Eppendorf Microinjector 5246 and MIS-5000 Burleigh micromanipulator; injection pressure of 100 hPa, compensation pressure of 50 hPa, and injection time of 0.1 s. The injected volume was 1 nl/shot. Neurons were injected into the cytosolic area of the cell soma and 90% survive the microinjection of DTR for at least 16 days.

Astrocytes were injected with 0.3 nl/cell at an injection pressure of 50 hPa, compensation pressure of 30 hPa, and an injection time of 0.1 s. Astrocytes were injected in the cytosol. Approximately 50% human astrocytes survive the injection for at least 16 days.

Measurement of Cell Death by TUNEL

Neurons were fixed in fresh 4% paraformaldehyde/4% sucrose in PBS and permeabilized with 0.1% Triton X-100 in 0.1% sodium citrate. Cell death was detected by TUNEL (TdT-mediated dUTP Nick End Labeling) using the Cell Death Kit I (Roche Molecular Biochemicals) as described by the manufacturer. The percentage of neuronal cell death was determined by the ratio of the number of DTR-TUNEL-double-positive neurons over the total number of DTR-positive neurons. The number of DTR positive neurons did not decrease with time indicating the retention of all apoptotic and non-apoptotic microinjected neurons on the coverslip.

Treatment with 17-β-estradiol, 17-α-estradiol, Testosterone Enanthate, Epitestosterone, Tamoxifen, Diethylstilbesterol or Ethinyl Estradiol

All were obtained from Sigma and dissolved as stock solutions in 100% ethanol. Dilutions of 1/1000 was made in culture media immediately before use. The media was changed with fresh solution every 48 hours. Controls received an equivalent amount of ethanol.

Treatment of Cells with Cycloheximide

Cycloheximide (Sigma) was made at 1 mg/ml in distilled-water and diluted at 5 and 20 μg/ml in culture media before treatment. To assess the efficiency of cycloheximide as an inhibitor of translation at these concentrations, neurons were labeled with 100 μCi/ml of ³⁵S-methionine (Easy Tag NEN-DUPONT) for 6 hours in the absence or presence of cycloheximide. Proteins were extracted in NP-40 lysis buffer, immunoprecipitated and separated by 10% polyacrylamide gel electrophoresis. To test the effect of cycloheximide on the neuroprotective effect of 17-β-estradiol, neurons were microinjected with R-Csp-6 and incubated with 10 nM 17-β-estradiol in the absence or presence of cycloheximide for 48 hours. To test the effect of cycloheximide on 17-β-estradiol induction of CIF, neurons were incubated with 10 nM 17-β-estradiol in the absence or presence of cycloheximide for 6 hours.

Protein Extracts of Treated Cells and Measurement of Caspase-6 Inhibitor Factor (CIF) Activity

After treatment, neuron proteins were extracted in caspase lysis buffer (50 mM Hepes pH 7.4, 0.1% CHAPS, 1 mM DTT, 0.1 mM EDTA) for 10 minutes on ice followed by microcentrifugation to remove insoluble material. Protein concentration was determined by bicinchoninic acid (BCA) assay (Pierce). Proteins (10 μg/100 μl assay) were added to 10 ng recombinant active caspase (Pharmingen or BioMol) in caspase assay buffer (20 mM Pipes, 30 mM NaCl, 10 mM DTT, 1 mM EDTA, 0.1% CHAPS, 10% sucrose pH 7.2) and 68.5 μM Ac-VEID-AFC for caspase-6, Ac-DEVD-AFC for caspase-3 and caspase-7, and Ac-IETD-AMC for caspase-8 (BioMol). The caspase-6 activity was measured at 37° C. every 2 minutes for 1 hour to determine the linear range of activity. Based on an AFC or AMC standard curve, the amount of released AFC or AMC was measured and the specific activity of the caspase determined as nmoles released AFC or AMC/pg protein/minute.

Statistics

Statistical evaluations of the difference between samples was done using a two-tailed t-test. Compared values are indicated in the legend of each figure.

Results

Neuronal Cell Death is Delayed by 17-β-Estradiol

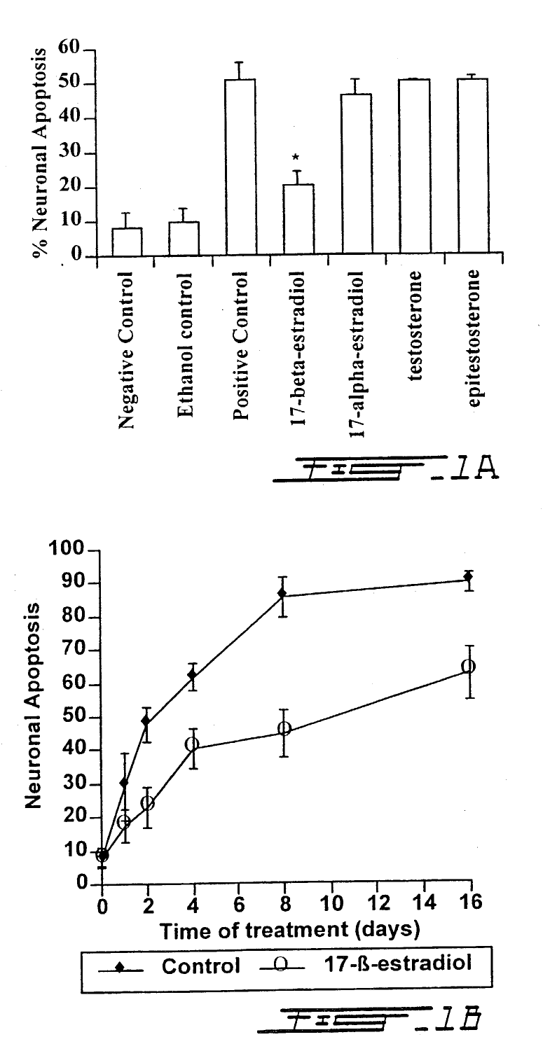

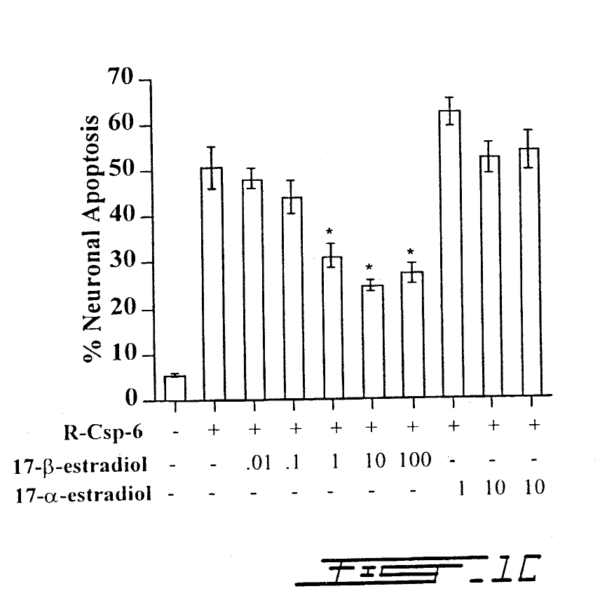

Caspase-6 is activated in serum deprived primary human neurons in culture (LeBlanc A C et al. (1999) J. Biol. Chem. 274:23426-23436). By direct microinjection of recombinant active caspase-6 (R-Csp-6), we have shown that caspase-6 induces apoptosis in primary cultures of human neurons (Zhang Y et al. (2000) J. Neurosci. 20:8384-8389). To determine if known neuroprotective agents can prevent R-Csp-6 mediated neuronal apoptosis, we treated the neurons microinjected with a lethal dose of 5 pg R-Csp-6/cell with 10 nM 17-β-estradiol (FIG. 1A). In 48 hours, R-Csp-6 induces apoptosis in 50% of microinjected neurons. 17-β-estradiol decreases the level of apoptosis to 20%. In contrast, 10 nM of the transcriptionally inactive estrogen, 17-α-estradiol, or androgens, testosterone, and epitestosterone do not protect significantly against R-Csp-6. These results indicate that the 17-β-estradiol prevents caspase-6-mediated neuronal apoptosis.

To determine if cell death is merely delayed or completely abrogated, a time study examined neuronal apoptosis of R-Csp-6 microinjected neurons incubated in the absence or presence of 10 nM 17-β-estradiol (FIG. 1B). The 17-β-estradiol confers 50% protection against caspase-6 until 8 days. However, increasing numbers of cells undergo apoptosis in time indicating that cell death is only delayed by 17-β-estradiol and not completely inhibited.

To determine if physiological concentrations of 17-β-estradiol protect against caspase-6-mediated cell death, various concentrations of 17-β-estradiol and 17-α-estradiol were tested (FIG. 1C). The. results show significant protection against caspase-6 mediated apoptosis with 1 to 100 nM but not 0.01 or 0.1 nM 17-β-estradiol. Since the normal pre-menopausal levels of estrogen in plasma are 2 nM, our results show that physiological concentrations of 17-β-estradiol can protect neurons against active caspases.

17-β-estradiol Induces an Inhibitory Factor of Caspase-6 Activity in Human Neurons

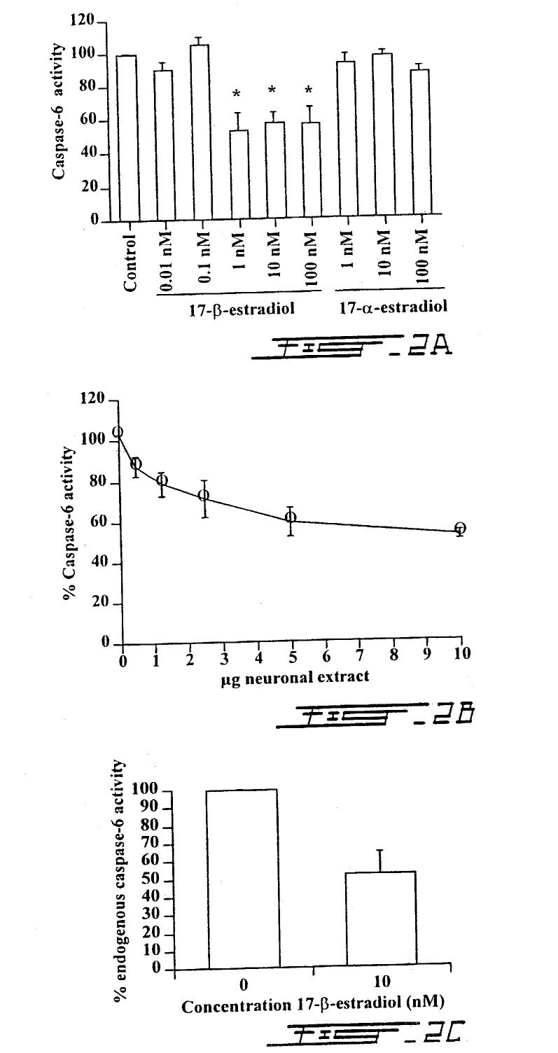

To determine if the 17-β-estradiol-mediated neuroprotective effect against caspase-6 is directly preventing R-Csp-6 activity or activating a survival pathway that interferes downstream of caspase-6, we tested 17-β-estradiol-treated neuronal extracts on R-Csp-6 activity in vitro. Neuronal extracts from 17-β-estradiol-treated neurons inhibit the activity of R-Csp-6 by approximately 40-60% (p<0.02) compared to 17-β-estradiol (FIG. 2A). The caspase inhibitory activity is induced with physiological 1 nM concentrations of 17-β-estradiol and does not change significantly with 10 or 100 nM concentrations (FIG. 2A). In contrast, neither 1, 10, or 100 nM 17-α-estradiol significantly inhibit active caspase-6. Hormones added directly to the R-Csp-6 assay in absence of neuronal extracts do not alter the activity of caspase-6. The profile of caspase inhibition at different doses of 17-β-estradiol parallels that of the inhibition of neuronal apoptosis (compare FIG. 1C with FIG. 2A). Increasing amounts of 17-β-estradiol-treated neuronal extracts parallel increasing CIF activity in vitro indicating a dose-dependent inhibition of R-Csp-6 (FIG. 2B). In addition, 17-β-estradiol treatment of neurons inhibits endogenous caspase-6 activity (FIG. 2C). These results indicate that physiological levels of 17-β-estradiol induce a neuronal caspase inhibitory factor (CIF) that acts directly on the active caspase-6.

CIF is Induced Within 10 Minutes of Neuronal Exposure to 17-β-Estradiol

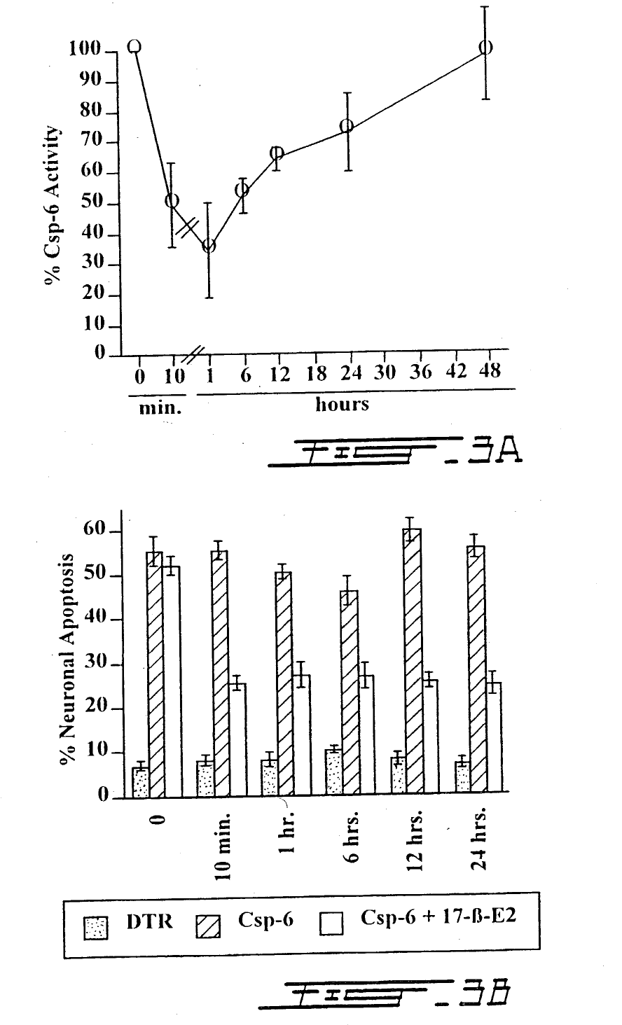

A time response curve of CIF activity shows that 17-β-estradiol induces CIF activity within 10 minutes of exposure to neurons and maximally at 1 hour of exposure (FIG. 3A). Thereafter, the neurons lose some of the activity indicating strong regulation of CIF activity. To determine if continued exposure to 17-β-estradiol is required for neuroprotection, we treated caspase-6 microinjected neurons with 17-β-estradiol for various times, washed the hormone away and incubated until 48 hours. We find that neuronal apoptosis is decreased to maximal levels when cells are exposed for only 10 minutes to 17-β-estradiol (FIG. 3B). Longer treatment of the neurons with 17-β-estradiol does not alter the level of neuroprotection. These results indicate that the induction of CIF within 10 minutes is rapid and sufficient to protect neurons against caspase-6.

De novo Protein Synthesis is not Required for 17-β-Estradiol Induction of CIF in Neurons

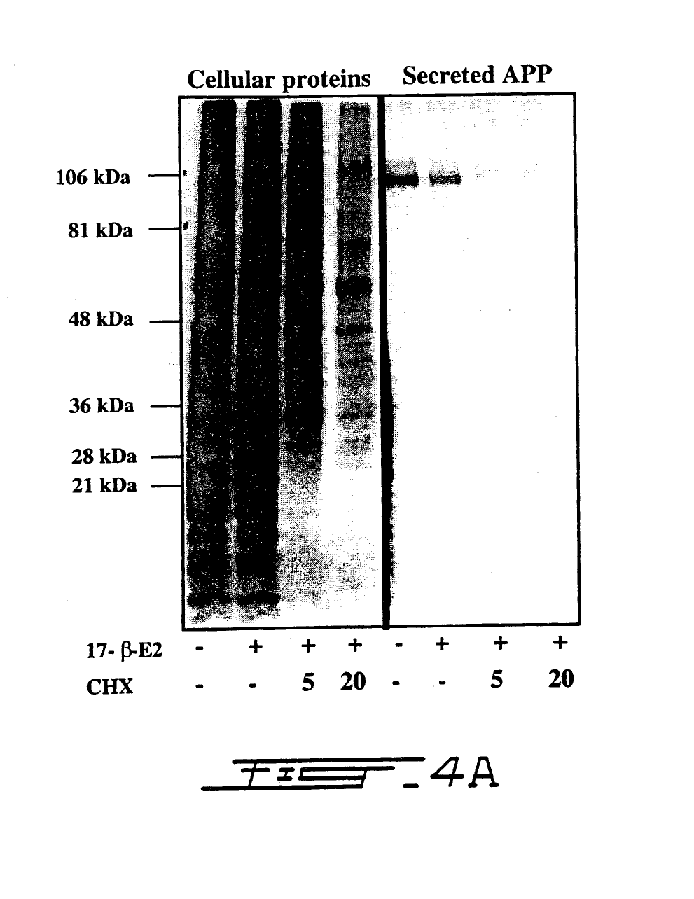

The rapidity with which 17-β-estradiol induces CIF suggests that CIF activity does not require de novo protein synthesis. To conclusively determine if 17-β-estradiol can induce CIF without protein translation, we treated the neurons with 5-20 μg/ml of cycloheximide in the presence of 10 nM 17-β-estradiol. While these doses of cycloheximide greatly inhibit protein translation in neurons (FIG. 4A), cycloheximide has no effect on the 17-β-estradiol-mediated neuroprotection (FIG. 4B) or induction of CIF (FIG. 4C). These results show that the activation of CIF does not require de novo protein synthesis.

CIF is not Acting Through Oxidation of the Active Cysteinyl Site of Caspase-6

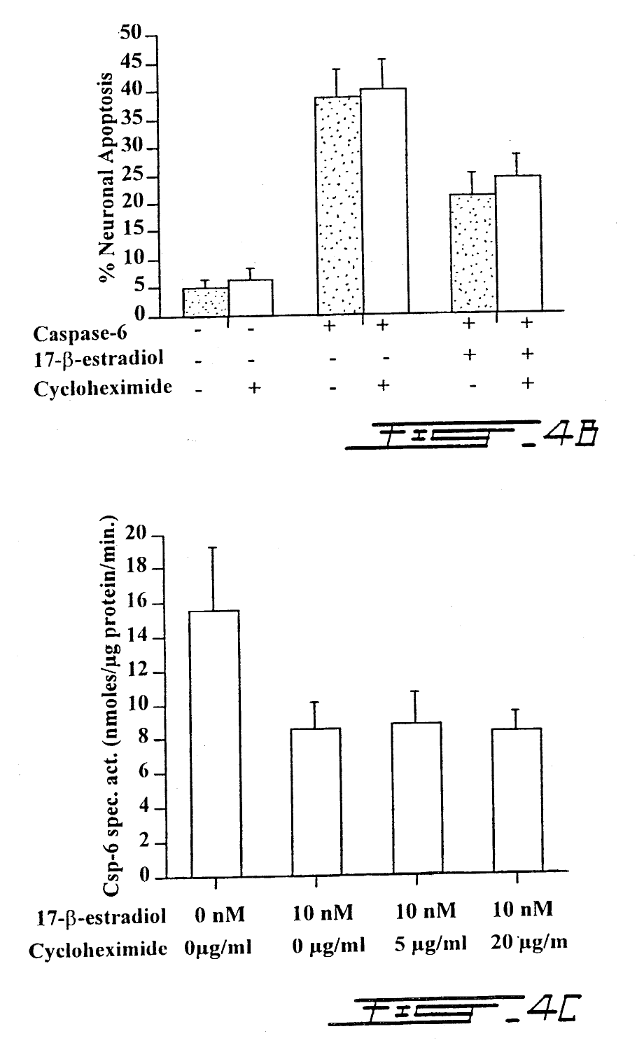

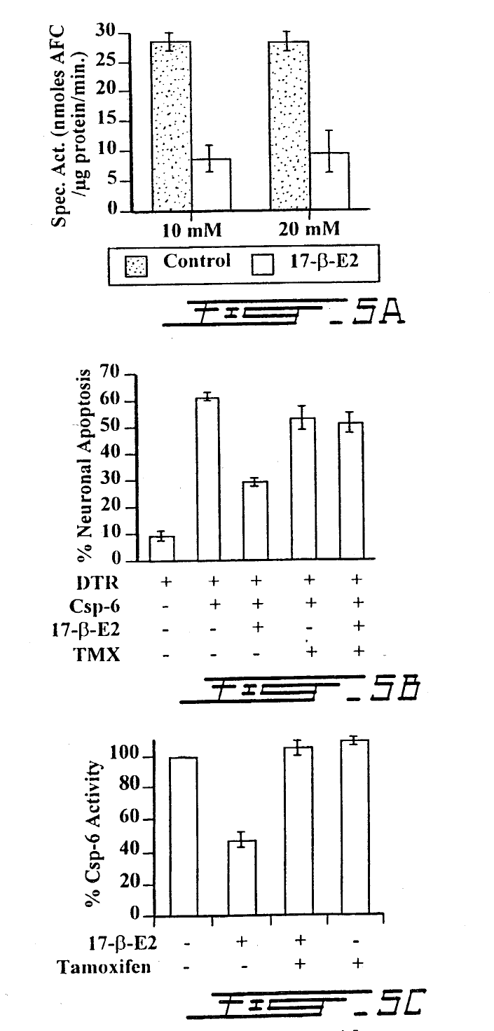

Of the known inhibitors of caspase activity, only nitric oxide that is induced by estrogens was a potential candidate for inhibition of active caspase-6. Nitric oxide can nitrosylate active caspases thus inhibiting their activity. The inhibitory activity of nitric oxide is reversible with 20 mM DTT. Similarly, selenite has been found to oxidize caspase active sites, a process that is also reversible by DTT. The caspase assays already contain 10 mM DTT and increasing the amount to 20 mM did not alter the caspase inhibitory activity indicating that CIF is not acting through an oxidative mechanism (FIG. 5A).

Anti-Estrogen, Tamoxifen, Inhibits CIF Induction by 17-β-Estradiol

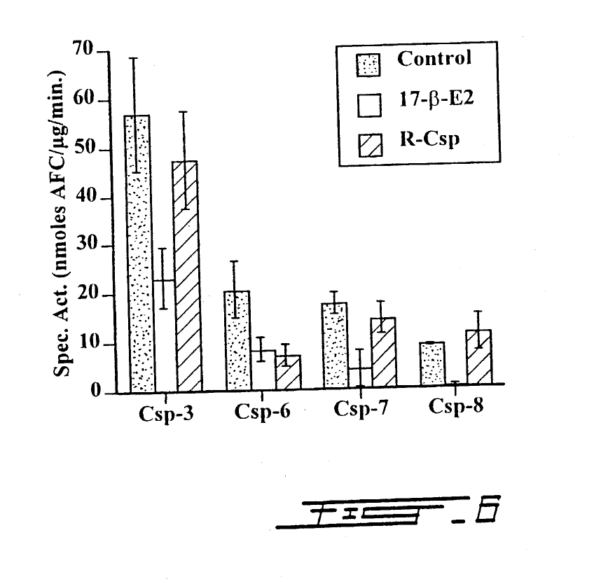

To determine if CIF activity is induced by estrogen receptors, we assessed the ability of estrogen receptor antagonist, tamoxifen, to block 17-β-estradiol induced CIF activity. Tamoxifen efficiently blocked both the neuroprotective function of 17-β-estradiol against caspase-6 (FIG. 5B) and CIF activation (FIG. 5C). These results indicate that estrogen receptors mediate CIF induction. CIF also inhibits caspase-3, 7, and 8 To determine if CIF activity is specific to caspase-6, we tested the 17-β-estradiol treated neuronal extracts for CIF activity on recombinant caspase-3, 7, and 8 (FIG. 6). Note that only caspase-6 activity is enhanced with the addition of protein extract. However, all caspases are inhibited by CIF. The inhibitory effect is stronger on caspase-7 (70%) and caspase-8 (90%) and similar for caspase-3 and caspase-6 (˜50%). These results show that CIF is not specific to caspase-6 and can inhibit other active caspases.

Cell Type Specificity of 17-β-Estradiol Mediated CIF Activity

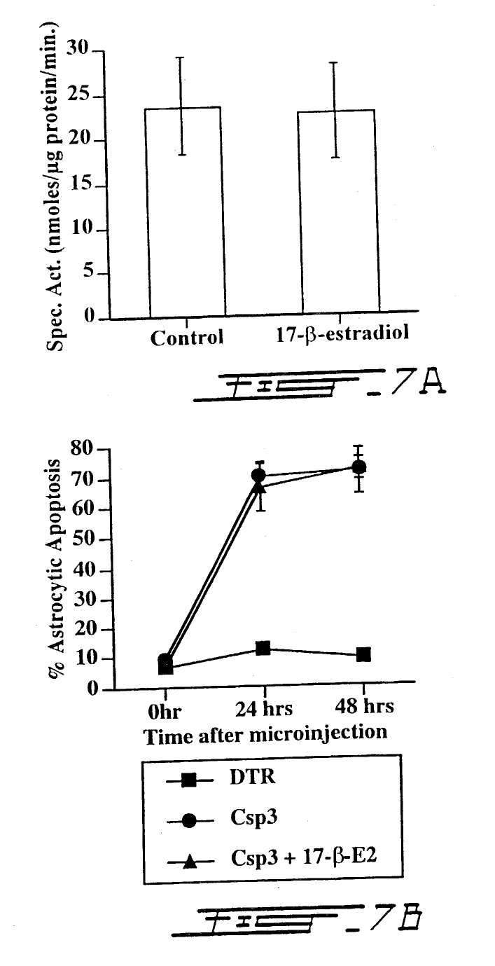

To determine if CIF can be activated in other cell types of the CNS, we treated astrocytes with 10 nM 17-β-estradiol for 6 hours and tested CIF activity in vitro (FIG. 7A). In contrast to neurons, CIF is not activated in astrocytes treated with 17-β-estradiol despite the presence of estrogen receptors in astrocytes (Woolley CS (1999) Curr. Opin. Neurobiol. 9:349-354). Since caspase-6 cannot induce cell death in astrocytes but caspase-3 does (Zhang Y et al. (2000) J. Neurosci. 20:8384-8389), we verified the ability of 17-β-estradiol to protect against caspase-3-mediated astrocytic cell death in the absence of CIF production. 17-β-estradiol could not prevent casapse-3 mediated astrocytic cell death (FIG. 7B). These results support the hypothesis that CIF is required for 17-β-estradiol inhibition of caspase-mediated cell death.

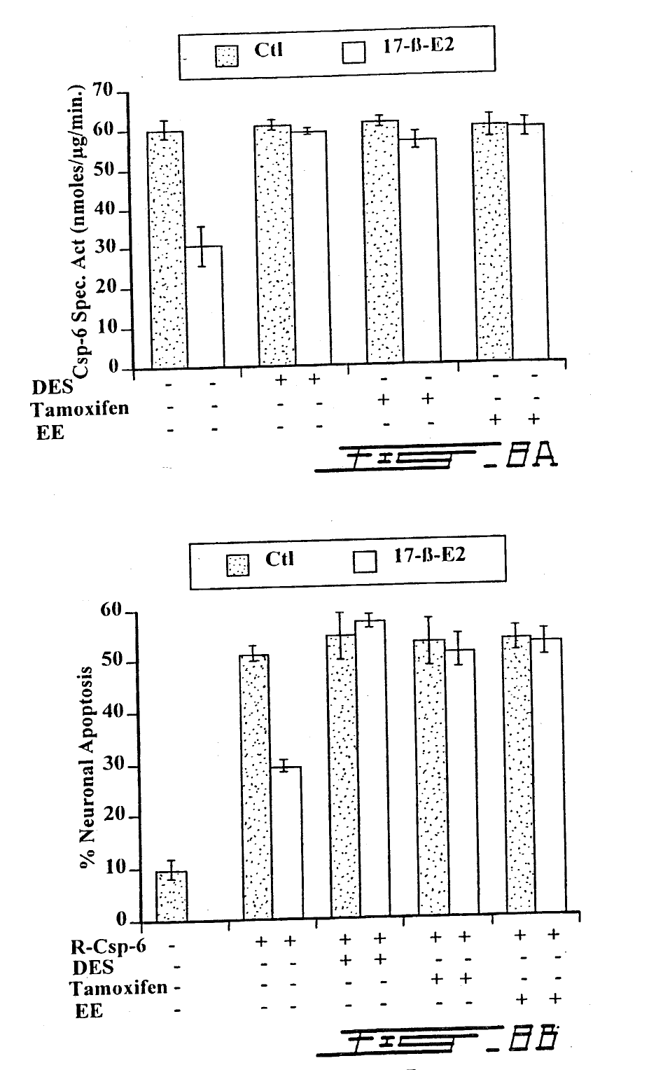

To determine if other estrogenic compounds can induce CIF, we treated human primary neurons with 10 nM 17-β-estradiol, diethylstilbesterol (DES), or ethinyl estradiol (EE). Neither DES nor EE induce CIF (FIG. 8A) nor induce neuroprotection (FIG. 8B) in neurons. However, addition of DES or EE to 17-β-estradiol inhibits 17-β-estradiol induction of CIF and neuroprotective effect against caspase-6. Similarly, the estrogen receptor antagonist, tamoxifen, inhibits 17-β-estradiol effect. We conclude from these experiments that DES and EE act as antagonists to CIF induction and neuroprotection against caspase-6. While tamoxifen indicates a classical estrogen receptor response, the results with DES and EE are unexpected and suggests that CIF is induced in a highly specific manner by 17-β-estradiol.

Discussion

Caspases are implicated in a broad range of central nervous system (CNS) diseases such as neurodegeneration, trauma and stroke (Thornberry N A (1999) Cell Death and Differentiation 6:1023-1027) Once activated, caspases induce irreversible molecular proteolytic cascades that result in cell death. In neurodegenerative diseases, considerable evidence supports a role for caspases in the pathogenesis of Alzheimer's disease, amyotropic lateral sclerosis and ischemia. Although caspase activation is secondary in these diseases or disorders, there is a strong interest in preventing caspase activation in order to avoid the loss of indispensable neurons and in the hope that survival of this cell type will allow treatment of the disease.

In accordance with the present invention, we demonstrate that 17-β-estradiol induces a caspase inhibitory factor (CIF) in primary cultures of human neurons. We find that 17-β-estradiol protects neurons against caspase-6 mediated cell death. The effect is highly specific since the transcriptionally inactive analogue, 17-α-estradiol, and androgens, testosterone or epitestosterone do not protect neurons against caspase-6 mediated neuronal apoptosis. Neuronal protein extracts from 17-β-estradiol, but not from 17-α-estradiol, testosterone or epitestosterone, inhibit recombinant active caspase-6 in vitro. These results indicate that 17-β-estradiol induce a caspase inhibitory factor (CIF). In contrast, 17-β-estradiol cannot protect against caspase-mediated astrocytic cell death nor induce CIF in astrocytes. Therefore, a clear correlation exists between 17-β-estradiol neuroprotection and CIF activity. We propose that CIF represents an endogenous caspase inhibitor that is induced by 17-β-estradiol, can inhibit active caspases in neurons and delay neuronal cell death.

The induction of CIF and neuroprotection occurs at 1 nM physiological concentrations of 17-β-estradiol. This feature indicates that induction of CIF represents an underlying molecular mechanism of neuronal protection by 17-β-estradiol. Epidemiological studies have shown that decreasing levels of estrogen increase the risk for Alzheimer's disease and hormone replacement therapy with estrogen (HRT) offers some protection against Alzheimer's disease if taken prophylactically (Mulnard R A et al. (2000) JAMA 283:1007-1015). Evaluation of the potential role of estrogen on neurons (Woolley C S (1999) Curr. Opin. Neurobiol. 9:349-354) identified that estrogen enhances neuritic outgrowth and survival, upregulates brain derived neurotrophic factor, nerve growth factor and epidermal growth factor, and reverses the behavioral and biochemical changes in ovariectomized rats. Estrogen modulates p53 activity and cell fate, and the expression of Bcl-2 proteins. Others propose that estrogen acts as an anti-oxidant although it is unlikely that physiological levels of estrogen will have antioxidant activity. Our results show a novel action of 17-β-estradiol against caspases.

The two known estrogen receptors ER-α and ER-β, are expressed in neurons and astrocytes. We show that estrogen receptor antagonist, tamoxifen, prevents 17-β-estradiol-mediated neuroprotection and induction of CIF indicating that 17-β-estradiol acts through its receptor. At this time, we do not know if 17-β-estradiol acts through the ER-α ER-β receptors. Since both receptors are expressed in neurons and astrocytes but CIF is only induced in neurons, CIF is either induced through an unknown exclusively neuronal receptor or the pathway regulating CIF activity is absent in astrocytes.

Estrogens modulate cellular activities through receptor-mediated nuclear gene transcriptional activation or through non-genomic mechanisms via signal transduction pathways (reviewed by Woolley C S (1999) Curr. Opin. Neurobiol. 9:349-354). Clearly, 17-β-estradiol induction of CIF occurs through a genomic-independent pathway since de novo protein synthesis is not required for CIF activity. The fact that induction of CIF occurs rapidly within 10 minutes and does not require de novo protein synthesis indicates that 17-β-estradiol may induce a signal transduction pathway leading to the activation of CIF.

CIF is also a broad spectrum inhibitor of caspases since it inhibits caspase-3, 6, 7, and -8. We could not verify if 17-β-estradiol can also prevent neuronal apoptosis mediated through other caspases since primary human neurons are selectively susceptible to caspase-6 (Zhang Y et al. (2000) J. Neurosci. 20:8384-8389). We believe that CIF represents a novel caspase inhibitor. Natural endogenous inhibitors of caspase-6 are unknown at this time. Caspase inhibitors can be grouped in two categories: Bcl-2, phosphorylation and mimic or decoy molecules inhibit the activation of pro-caspases while Crm A, p35, IAPs, phosphorylation and nitrosylation inhibit the active form of caspases (Ekert P et al. (1999) Cell Death and Differentiation 6:1081-1086). Since CIF inhibits the active form of caspases, the first group of inhibitors is eliminated as potential CIF candidates. Within the second group, p35 can inhibit caspase-1,-3,-6,-7,-8, and -10 but is absent in our system. Members of the IAP family, X-IAP, c-IAP-1, and c-IAP-2, inhibit caspases-3, -7, and -9 by direct interaction with the caspases but none can inhibit caspase-6. Nitric oxide nitrosylation and selenite oxidation of the cysteine residues of caspase-3 inhibit activity and the inhibition is reversible by 20 mM DTT. However, since R-Csp-6 activity is not restored with increasing concentrations of reducing agents, it is clear that neither nitrosylation or oxidation plays a role in CIF activity. Phosphorylation of pro-caspase-9 or the large subunit of caspase-9 by serine/threonine kinase, Akt, inhibits caspase-9 activity. Akt phosphorylation is unlikely since caspase-6 lacks consensus Akt phosphorylation motifs. It is however possible that other kinases are activated and phosphorylate caspase-6. Therefore, we believe that CIF is a novel 17-β-estradiol regulated inhibitor.

There is considerable interest in generating synthetic caspase inhibitors for treatment of caspase-mediated apoptosis. Synthetic peptide inhibitors are made based on the specificity of caspases for four amino acid substrates with an obligatory aspartic acid at P1. Classification of caspases have been established based on substrate preference. While synthetic caspase inhibitors hold great promise for many diseases, there is a concern that they may not target specific cell types and in the case of the brain could predispose to tumorigenicity rather than simply prevent neuronal cell death by caspase inactivation. Therefore, natural endogenous inhibitors may offer a more selective approach to therapeutic treatment. CIF is particularly interesting since it provides a broad spectrum caspase inhibitor that is specific to neurons in brain and may be useful in inhibiting caspase mediated apoptosis in a variety of diseases.

In conclusion, we have identified a novel and unsuspected mechanism by which estrogen protects human neurons against cell death by inducing a caspase inhibitory factor (CIF). CIF could prevent caspase-mediated cell death in neurodegenerative diseases.

While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth, and as follows in the scope of the appended claims.

What is claimed is:

1. A caspase inhibitory factor (CIF) which comprises a factor having the identifying characteristics of a factor endogenous to a human primary culture of neurons and endogenous to a human breast cancer cell line, MCF-7, and wherein said CIF being inducible by 17-β-estradiol and being capable of preventing apoptosis and/or synaptic degeneration.

2. The caspase inhibitory factor (CIF) of claim 1, wherein said preventing of apoptosis and/or of synaptic degeneration is effected by inhibiting at least one caspase.

3. The caspase inhibitory factor (CIF) of claim 2, wherein said caspase is caspase-3, -6, -7 or -8.

4. A drug screening assay for potential neuronal inhibitors of caspases, which comprises using a caspase-inhibitory-factor (CIF) of claim 1 to test for compounds capable of activating CIF.

5. A drug screening assay for potential compounds for the treatment of neurodegenerative diseases and metabolic bone diseases, which comprises using caspase-inhibitory-factor (CIF) of claim 1 to test for compounds capable of activating CIF.

6. The assay of claim 5, wherein said metabolic bone diseases are osteomalacia, osteoporosis, osteopetrosis or Paget's disease.

7. The assay of claim 5, wherein said neurodegenerative diseases are selected from the group consisting of Parkinson's, Alzheimer's disease, neuronal loss associated with dementia, amyotropic lateral sclerosis (ALS), cerebellar degeneration, ischemia (stroke), traumatic injuries, prion diseases and Huntington disease.

8. A drug screening assay for potential compounds for the treatment of estrogen responsive cancers, which comprises using caspase-inhibitory-factor (CIF) of claim 1 to test for compounds capable of inhibiting CIF.

9. The assay of claim 8, wherein said estrogen responsive cancers is breast and uterine cancer.

10. A method for the treatment of neurodegenerative diseases and metabolic bone diseases, which comprises administering an effective amount of a compound capable of activating CIF.

11. The method of claim 10, wherein said metabolic bone diseases is osteomalacia, osteoporosis, osteopetrosis or Paget's disease.

12. The method of claim 10, wherein said neurodegenerative diseases are selected from the group consisting of Parkinson's, Alzheimer's disease, neuronal loss associated with dementia, amyotropic lateral sclerosis (ALS), cerebellar degeneration, ischemia (stroke), traumatic injuries, prion diseases and Huntington disease.

13. A method for the treatment of estrogen responsive cancer which comprises administering an effective amount of a compound capable of inhibiting CIF.

14. The method of claim 13, wherein said estrogen responsive cancer is breast and/or uterine cancer.

15. A method for the protection against apoptosis in estrogen responsive tissues in a patient, which comprises administering an effective amount of a compound capable of activating CIF to the same level as 17-β-estradiol.

16. The method of claim 15, wherein said estrogen responsive tissues is selected from the group consisting of neurons and bone.

17. A method of determining the ability of cells to become malignant, which comprises determining whether the presence of estrogen in said cells increases the activity of CIF.

18. A method of diagnosis of a disease associated with apoptosis, which comprises detecting and/or quantitating CIF activity.

19. A drug screening assay for potential estrogen capable of protecting against apoptosis, which comprises using a caspase-inhibitory-factor (CIF) of claim 1 to test for compounds capable of activating CIF.
